(19) United States
(12) Patent Application Publication
(10) Pub. No.: US 2006/0278239 A1
(43) Pub. Date: Dec. 14, 2006

(54) Kissing shield game and method of use thereof

(76) Inventor: Deloris Wood, Salem, MO (US)

Correspondence Address: DUNLAP, CODDING & ROGERS P.C., PO BOX 16370, OKLAHOMA CITY, OK, 73113, US

(21) Appl. No.: 11/443,336

(22) Filed: May 30, 2006

Related U.S. Application Data
(63) Continuation-in-part of application No. 10/892,909, filed on Jul. 16, 2004, now Pat. No. 7,086,646.
Application No. 10/892,909 is a continuation of application No. 10/195,772, filed on Jul. 12, 2002, now Pat. No. 6,789,799.
Application No. 10/892,909 is a continuation-in-part of application No. 09/466,004, filed on Dec. 17, 1999, now Pat. No. 6,293,280.
(60) Provisional application No. 60/304,980, filed on Jul. 12, 2001.

(51) Int. Cl. A63F 3/00; A63F 3/02; A61F 11/00; A61C 5/14

(52) U.S. Cl. 128/857; 128/859; 273/236; 273/244; 273/243; 273/259; 273/277

(57) ABSTRACT

The present invention is directed to a kissing shield game and method teaching safe affection to at least one person playing the kissing shield game. The kissing shield game includes a plurality of removable pegs, a playing surface, a gaming member, a plurality of frames and a plurality of thin, flexible membranes forming a finger kissing shield, and a resettable timer. The playing surface has a plurality of holes arranged in a predetermined pattern with each of the plurality of holes being capable of receiving at least one of the plurality of removable pegs. Each side of the gaming member has a different color associated therewith as well as a predetermined set of alphanumeric symbols associated therewith. The finger kissing shield is used when a selected person or object determined by a player is kissed so that the player learns safe affection and the proper use of the finger kissing shield through repetition.

### CROSS REFERENCE TO RELATED APPLICATIONS

This application is a continuation-in-part of U.S. Ser. No. 10/892,909 which is a continuation of U.S. Ser. No. 10/195,772, filed Jul. 12, 2002, entitled “KISSING SHIELD GAME AND METHOD OF USE THEREOF”, now U.S. Pat. No. 6,789,799, which claims benefit under 37 CFR 119(e) of U.S. Provisional No. 60/304,980, filed Jul. 12, 2001, entitled “KISSING SHIELD GAME;” and is also a continuation-in-part of U.S. Ser. No. 09/466,004, filed Dec. 17, 1999, entitled “KISSING SHIELD AND METHOD OF USE THEREOF,” now U.S. Pat. No. 6,293,280, the contents of all are hereby incorporated herein in their entirety by reference.

### STATEMENT REGARDING FEDERALLY SPONSORED RESEARCH OR DEVELOPMENT

Not applicable.

### BACKGROUND OF THE INVENTION

1. Field of the Invention

The present invention relates generally to a board game, and more particularly, but not by way of limitation, to a board game for teaching a person to practice safe affection utilizing a finger kissing shield and playing pieces.

2. Description of Background of Invention

There is a growing awareness of the seriousness of diseases, like Acquired Immunodeficiency Syndrome (AIDS), being spread and the need for protection of those not exposed to such diseases. At times, there has been hysteria among parents and other students, who are afraid their children or they themselves will become infected from classroom and playground contact, when students with AIDS or the Human Immunodeficiency Virus (HIV) have attended school. Also, persons who carry the herpes virus sometimes have lip sores which are usually not distinguishable from an ordinary canker sore or a fever blister by a lay person.

It is customary when we kiss to come in contact with another person's lips, and in certain cultures, to follow with a kiss on the skin of each cheek; thus germs can be passed from one person to another. In keeping with one aspect of the invention, if casual contact is necessary and a kiss is appropriate, one can protect oneself from the germs present in saliva or other secretions which might be transmitted from kissing by using a kissing shield.

The present invention proposes using a device in which a flexible membrane is used as a kissing shield to lessen ones chances of becoming infected by disease from casual contact. In the alternative, if a person is infected, the chances of transferring the infectious disease from one person to another could be reduced by use of a thin, resilient flexible, impervious membrane, preferably selected from the class of polyethylene, vinyl, and polypropylene materials, stretched over a frame or holder. This would lessen the spread of bodily fluids from one person to another when kissing with the end result of preventing the spread of viruses and diseases, such as canker sores, fever blisters, and AIDS until there is a cure and prevention of the diseases. The advantages of a kissing shield over regular kissing will become apparent on consideration of the following specification and the accompanying drawings wherein there is disclosed a preferred embodiment.

The kissing shield has both social and health benefits, if basic precautions, such as those one would engage in while using a condom to practice “safe sex”, or as a dentist would use when he dons rubber gloves to prevent bodily fluids, such as blood and saliva from his patient, from spreading to his hands and thereby infecting him, are used. The kissing shield can be economically mass produced so that it could be easily disposed of after kissing a person and replaced with a new one.

The kissing shield is for people who desire to be cautious when in contact with another person as they kiss. Use of the kissing shield is convenient and practical. However, like most items we use when we must alter our habits, education is an important step. The kiss is one of the first forms of affection that we display to another. It seems only natural that we would start at a fundamental level and teach “safe kissing” before we teach “safe sex”.

The kissing shield, if handled properly, will help people who want to do whatever they can while kissing to practice “preventive medicine” and ensure that diseases are not passed from one person to another by proper sanitation or cleanliness of one or both parties. A person who might have a disease and a person who does not want to get the disease or a person who is being protected would take precautionary moves to help prevent the spread of diseases, such as AIDS, by first practicing “safe kissing”. A kissing shield is for casual kissing. It can be used especially by a politician who kisses babies.

It is therefore a primary object of the invention to provide a simple, inexpensive kissing shield to be used when kissing mouth-to-mouth or mouth-to-cheek thereby avoiding the necessity for skin contact with the person to whom affection is intended. It is another object of the invention to provide a means for removing the hesitancy a user may have in kissing another individual without sacrificing the effectiveness of the kiss. It is a further object of the invention to provide a means of preventing the transmission of germs or viruses from saliva or other secretions and the transfer of lipstick or other cosmetics when individuals are engaged in kissing. It is another object of the invention to provide a shield that is to be worn on a person's fingers. It is another object of the invention to provide a shield which is economical in construction, such that the device can be used once and thereafter disposed of.

In addition, board games have traditionally been used as a form of recreational activity. The typical board game has a playing surface which provides the means on which the board game is played. Additionally, the board game is provided with accessories in the form of instruction cards, game pieces, gaming member, etc. The playing of the board game is dictated by a set of rules or instructions which refer to the movement of selected accessory parts around the playing surface. Board games are traditionally played by more than one person either as a group of individuals or in teams of defined size and are enjoyed by some families and groups as a form of recreational activity.

However, board games do not solely function as an alternative recreational activity. Many board games provide a teaching means to improve a person's general knowledge and education. To this end, a need exists for a board game utilizing the kissing shield that teaches safe affection. It is to such a board game that the present invention is directed.

### SUMMARY OF THE INVENTION

The present invention is directed to a kissing shield game and method for teaching safe affection to at least one person playing the kissing shield game. The kissing shield game includes a plurality of removable pegs; a playing surface; a gaming member; a plurality of thin, flexible membranes; and a resettable timer.

The playing surface has a plurality of holes. Each of the plurality of holes is capable of receiving at least one of the plurality of removable pegs which may be colored. The plurality of holes are arranged in a predetermined pattern. The gaming member has at least two sides. Each of the sides of the gaming member has a different color associated therewith as well as a predetermined set of alphanumeric symbols associated therewith. Each color associated with one of the sides of the gaming member is assigned a predetermined numerical value. In addition, at least one of the removable pegs is colored to correspond to at least one of the colors on at least one of the sides of the gaming member.

The plurality of thin, flexible membranes are supported by a person's fingers and are impervious to microorganisms. Each of the plurality of thin, flexible membranes prevents exchange of microorganisms between the person and an object while engaged in the act of kissing.

In another embodiment of the invention the thin flexible membrane is designed and configured such that the user inserts his/her middle and index fingers into the thin flexible membrane, positions the thin flexible membrane between the user's mouth and an object to be kissed and extends an index finger and a middle finger such that the thin flexible membrane is substantially stretched between the index finger and the middle finger and thereby prevents the exchange of microorganisms between the user and the object to be kissed.

In addition, the kissing shield game includes a set of rules which govern the function of the kissing shield game and the order and actions of a kissing shield game participant. A plurality of persons may be divided into a plurality of teams for playing the kissing shield game.

In use, at least one of the thin, flexible membranes is placed over the user's index and middle fingers so as to form a finger kissing shield. The gaming member is thrown along the playing surface so that one of the sides lands facing in an upward direction. A role playing adventure is determined within a predetermined time so as to teach safe affection in accordance with the alphanumeric symbol associated with the side of the gaming member facing in an upward direction. A selected person or object determined by the player is kissed with the finger kissing shield so that at least one person playing the kissing shield game learns safe affection and the proper use of the finger kissing shield through repetition. The end of the predetermined time is signaled by the resettable timer. A number is determined for sequential movement of at least one of the plurality of removable pegs in accordance with a predetermined number associated with the color of the side of the gaming member facing in the upward direction. If a role playing adventure is determined, then at least one of the removable pegs is moved in a forward direction along the plurality of holes arranged in the predetermined pattern in accordance with the predetermined number associated with the color of the side of the gaming member facing in an upward direction. However, upon the person playing the kissing shield game failing to determine the role playing adventure within a specified time, then at least one of the removable pegs is moved in a backward direction along the plurality of holes arranged in the predetermined pattern in accordance with the predetermined number associated with the color of the side of the gaming member facing in an upward direction. At least one of the removable pegs lands in at least one of the plurality of holes in accordance with the predetermined number associated with the color of the side of the gaming member facing in an upward direction. A kissing shield game winning player is determined based upon the first person playing the kissing shield game to advance in a forward direction along the plurality of holes in the predetermined pattern on the playing surface until the plurality of holes in the predetermined pattern ends.

The advantages and features of the present invention will become apparent to those skilled in the art when the following description is read in conjunction with the attached drawings and the appended claims.

### BRIEF DESCRIPTION OF THE SEVERAL VIEWS OF THE DRAWINGS

FIG. 1 shows a front plan view of a finger kissing shield placed over a user's index and middle fingers.

FIG. 2 is a side plan view of the finger kissing shield shown in FIG. 1.

FIG. 3 shows a front plan view of another embodiment of the present invention.

FIG. 4 shows a front plan view of another embodiment of the present invention.

FIG. 5 is a side plan view of an embodiment of the finger kissing shield wherein the finger kissing shield has a protuberance for the tongue.

FIG. 6 shows a flexible membrane constructed in accordance with the present invention.

FIG. 7 shows another embodiment of the flexible membrane constructed in accordance with the present invention.

FIG. 8 shows a top view of a playing surface of a kissing shield game, a gaming member, a plurality of removable pegs, and a resettable timer.

FIG. 9 shows a flat view of multiple sides of a gaming member used in the kissing shield game.

### DETAILED DESCRIPTION OF THE INVENTION

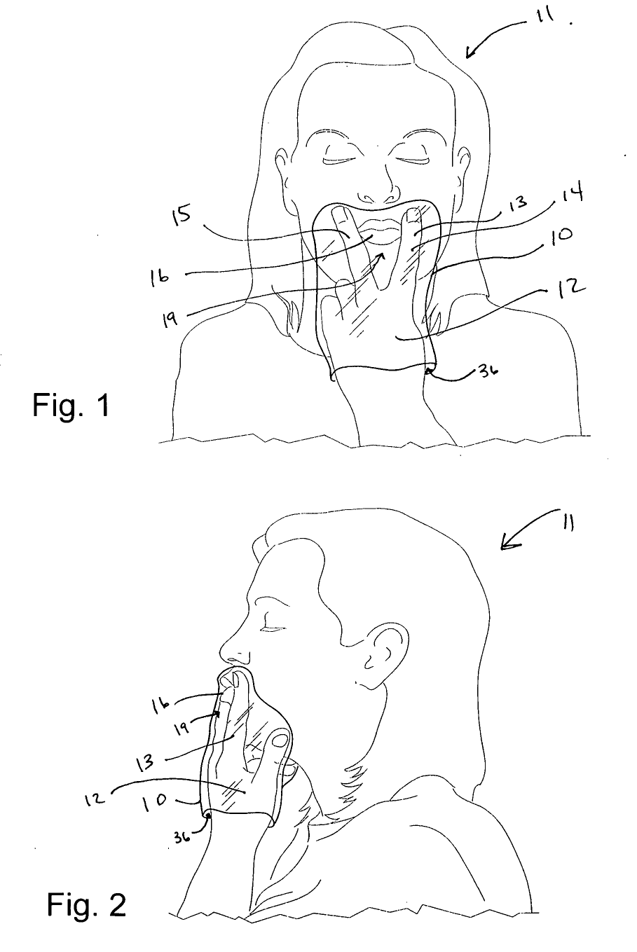

Referring now to the drawings wherein like reference characters represent like elements, FIG. 1 shows a thin, flexible, impervious membrane 10 placed over a user's 11 hand 12 and positioned in the appropriate location for use. Membrane 10 is closed on three sides; a fourth side remains open creating a pocket 36 so that the shield may be placed over the user's 11 hand 12 and over a user's 11 fingers 13. Membrane 10 may be selected from the group of thin, flexible, impervious materials, such as polyethylene, vinyl, and polypropylene. It will be appreciated that membrane 10 can be sized to be employed by a variety of users 11. As shown in FIG. 2, membrane 10 is adapted fit over a user's 11 hand 12 and fingers 13 and to surround the lower part of the face of the user 11. The membrane 10 can be held in position by a first finger 14 and a second finger 15 whereby the first finger 14 and the second finger 15 are spread to create a space 19 for a user's 11 lips 16 to deliver a kiss. The first finger 14 and the second finger 15 can be any finger 13, such as an index finger, a middle finger, a ring finger, a pinky finger, or a thumb. FIG. 1 shows an embodiment where the first finger 14 is an index finger and the second finger 15 is a middle finger.

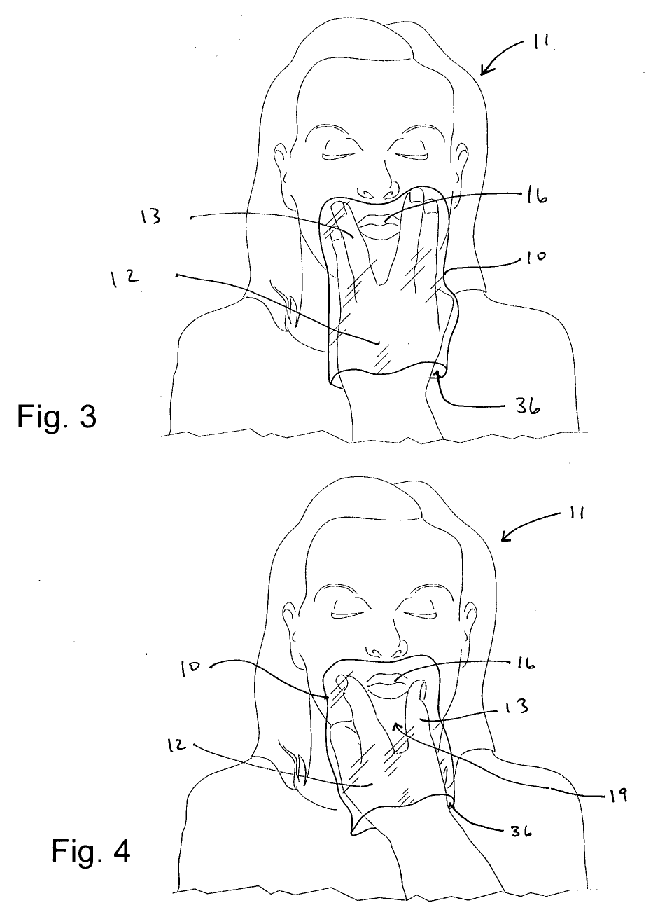

FIGS. 3 and 4 shows further embodiments of the present invention wherein the space 19 for the user's 11 lips 16 is created by using different fingers 13. FIG. 3 shows an embodiment wherein the first finger 14 is a middle finger and the second finger 15 is a ring finger. Alternatively, FIG. 4 shows an embodiment wherein first finger 14 is a thumb and the second finger 15 is an index finger.

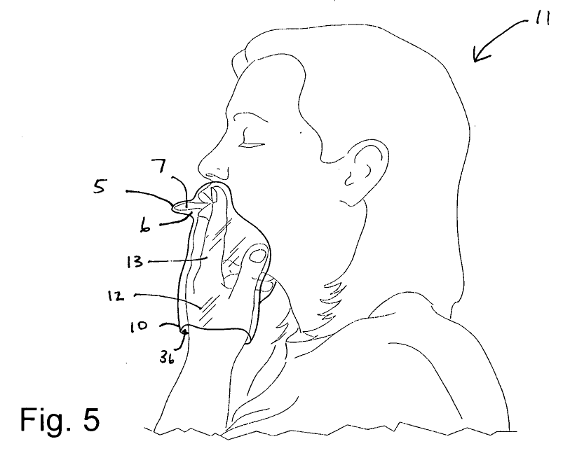

FIG. 5 shows thin, flexible, impervious membrane 10 having a protruding portion 5 located near the uppermost portion of membrane 10. Protruding portion 5 creates a cavity 6 into which the tongue 7 of a user 11 may be placed for a particular type of kissing.

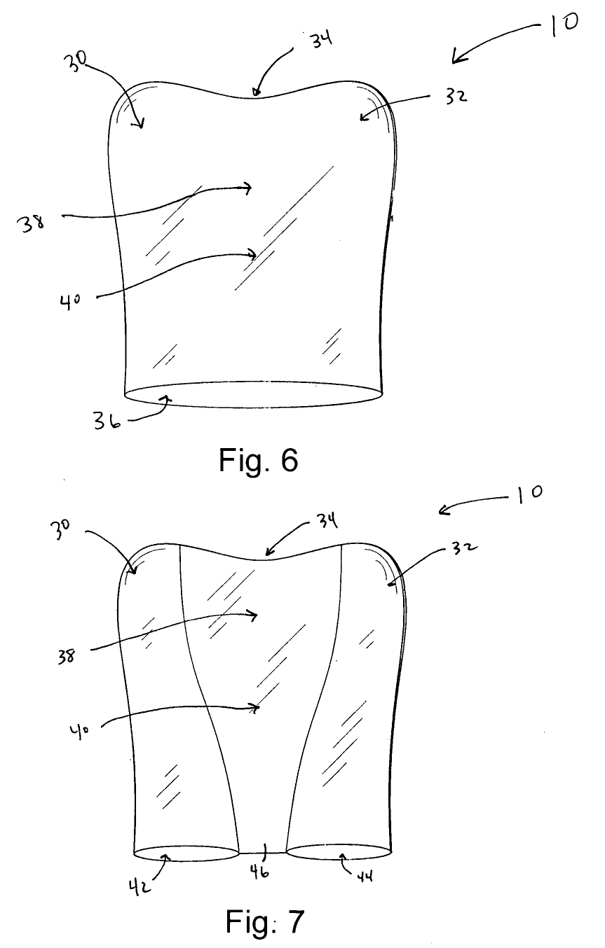

Referring now to FIG. 6, shown therein is the flexible membrane 10 constructed in accordance with the present invention. The flexible membrane 10 is a thin flexible membrane substantially impervious to microorganisms. The flexible membrane 10 includes a first cheek portion 30, a second cheek portion 32, a clearance 34, a chin portion 40, a central portion 38, and a pocket 36. Another embodiment, shown in FIG. 7, shows the flexible membrane 10 having a first pocket 42, a second pocket 44, and a single pane membrane 46. The first cheek portion 30 substantially covers a first cheek area of the user 11. The second cheek portion 32 substantially covers the second cheek area of the user 11. The clearance 34 is located between the first cheek portion 30 and the second cheek portion 32 near the top of the flexible membrane 10 configured, sized and designed to accommodate the nose of the user 11 and the thin flexible membrane 10 is properly positioned. The chin portion 40 of the thin flexible membrane 10 covers the chin of the user 11. The central portion 38 of the thin flexible membrane 10 covers the lips 16 and the mouth of the user 11 when the thin flexible membrane 10 is properly positioned for use. With respect to the embodiment of FIG. 6, the pocket 36 is sized, shaped, and configured so as to matingly receive the user's 11 hand 12 and fingers 13. In the embodiment shown in FIG. 7, the first pocket 42 is sized, shaped and configured so as to matingly receive a first finger 14 of the user 11 and the second pocket 44 is sized and configured so as to matingly receive a second finger 15 of the user 11. The first pocket 42 and the second pocket 44 are located and disposed such that the user's 11 first finger 14 can be disposed in the first pocket 42 and the user's 11 second finger 15 can be disposed in the second pocket 44. When the user 11 moves his first finger 14 and second finger 14 in opposing outward directions the central portion 38 of the thin flexible membrane 10, or the single pane membrane 46, covers the lips 16 and the mouth of the user 11 such that when the thin flexible membrane 10 is interposed between the user 11 and the object being kissed, the central portion 38 of the thin flexible membrane 10 prevents the exchange of microorganisms between the user 11 and the object during the act of kissing. It should be noted that the central portion 38 of the flexible membrane 10 can include a protruding portion 5 substantially configured to accommodate the tongue 7 of the user 11 such that the user's 11 tongue 7 is positionable in the protruding portion 5. The thin flexible membrane 10 can also include a message, design or pattern printed on the thin flexible membrane 10. The thin flexible membrane 10 can include a color, can be entirely colored, entirely clear, have an iridescent color or pearlescent color. Typically, the thin flexible membrane 10 is constructed of a synthetic material or a latex material such as polypropylene, vinyl, or polyethylene. The thin flexible membrane 10 can have a first side and a second side. The first side of the thin flexible membrane 10 can include a first color and the second side of the thin flexible membrane can include a second color.

In one embodiment of the invention, a kissing shield game includes a plurality of removable pegs 15, a playing surface 16, a gaming member 18,a plurality of thin, flexible membranes 10, and a resettable timer 100.

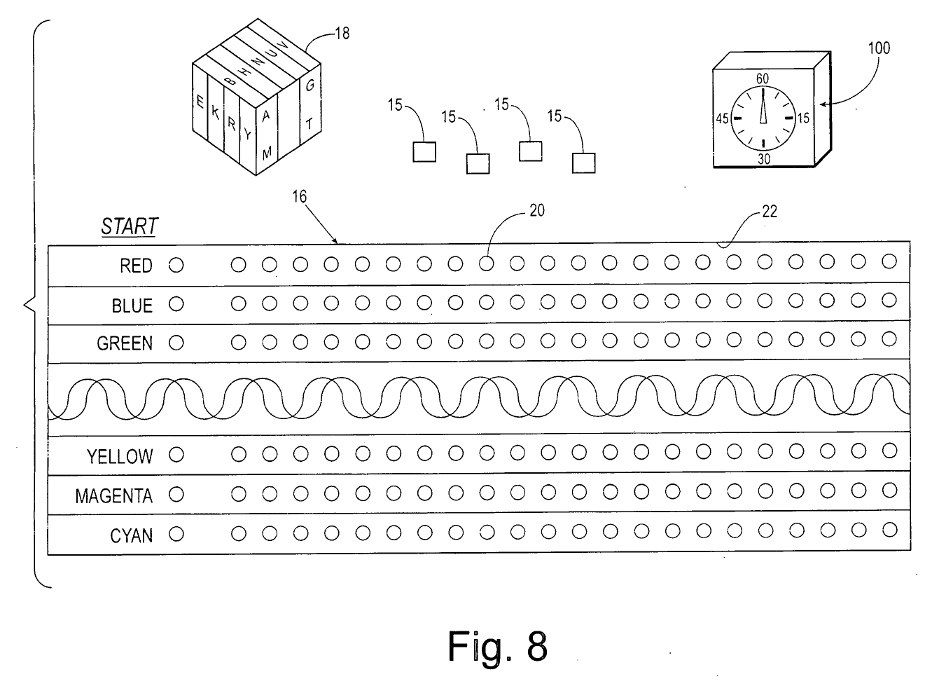

Referring now to FIG. 8, the playing surface 16 has a plurality of holes 20. Each of the plurality of holes 20 is capable of receiving at least one of a plurality of removable pegs 15 which may be colored. In addition, each of the plurality of flexible membranes 10 may also be colored. Any color may be used for the plurality of removable pegs 15, however, the color of each of the plurality of removable pegs 15 will preferably correspond to the color of the flexible membranes 10. Examples of colors that may be used for the removable pegs 15 and the plurality of flexible membranes 10 in the kissing shield game consist of red, blue, green, yellow, magenta, and cyan.

The plurality of holes 20 are arranged in a predetermined pattern. The predetermined pattern is preferably in a row 22, as shown in FIG. 6. However, it should be understood that the pattern may be any of a variety of patterns, such as diagonals, waves, or circles and as such should not be regarded as limiting.

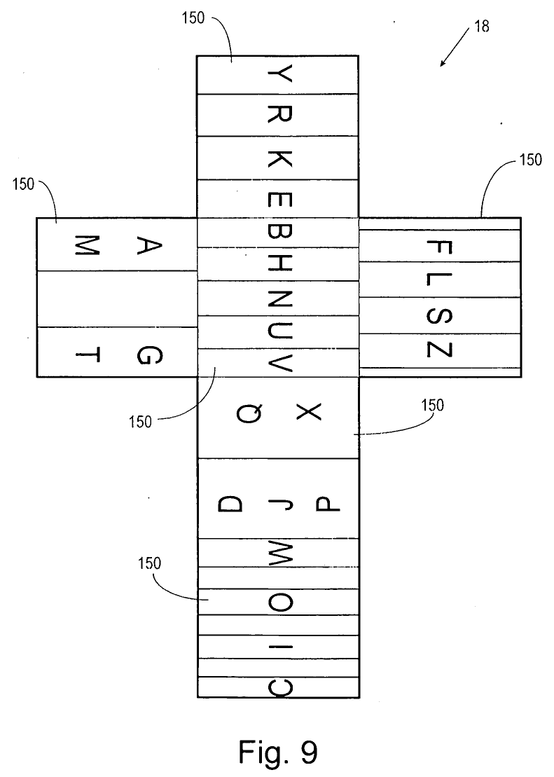

Shown in more detail in FIG. 9, the gaming member 18 has at least two sides. FIG. 9 shows the gaming member 18 having six sides, however, it should be understood that the gaming member may have any number of sides as long as there are at least two. Each of the sides of the gaming member 18 has a different color associated therewith as well as a predetermined set of alphanumeric symbols 150 associated therewith. Any color may be used in the kissing shield game. Examples of preferred colors include green, cyan, blue magenta, red, and yellow. Each color associated with one of the six sides of the gaming member 18 is assigned a predetermined numerical value for example, green-6, cyan-5, blue-4, magenta-3, red-2, and yellow-1. The predetermined numbers determine how many of the plurality of holes 20 the removable pegs 15 will be moved. In addition, each side of the gaming member 18 may have four or six different letters of the alphabet.

In use, the gaming member 18 is thrown by a player along the playing surface 20 so that one of the sides lands facing in an upward direction. The player determines a role playing adventure according to the side of the gaming member 18 that faces upward. The role playing adventure corresponds to one of the alphanumeric symbols 150 that are shown on the side of the gaming member 18 that is facing upward. For example, if the side of the gaming member 18 that is facing upward has a “c” showing, then the user must select a role playing adventure that begins with the letter “c”, e.g. kissing a cat. The player has a predetermined time to decide what sort of role-playing will be done so as to teach safe affection. A selected person or object determined by the player is kissed with the finger kissing shield so that at least one person playing the kissing shield game learns safe affection and the proper use of the finger kissing shield through repetition. So, for example, the player rolls the gaming member 18 and the gaming member 18 lands with one of the six sides facing upward that is green with the letters C, I, O and W. The player then determines a person or object beginning with one of the letters to kiss with the finger kissing shield.

The end of the predetermined time is signaled by the resettable timer 100 and a number is determined for sequential movement of at least one of the plurality of removable pegs 15 in accordance with a predetermined number associated with the color of the side of the gaming member 18 facing in the upward direction. If the player is successful in and acting out the role playing adventure, then the player may move the removable peg 15 in a forward direction along the plurality of holes 20 arranged in the predetermined pattern in accordance with the predetermined number associated with the color of the side of the gaming member 18 facing in an upward direction. Thus, if the numerical value six was assigned to the color green, green being the side of the gaming member 18 landing face up, then the player moves the removable peg 15 six holes forward along the plurality of holes 20. However, if the player does not determine and act out the role playing adventure within the specified time, then at least one of the removable pegs 15 (associated with the player) is moved in a backward direction along the plurality of holes 20 in accordance with the predetermined number associated with the color of the side of the gaming member 18 facing in an upward direction. So, the removable peg 15 will be moved six holes backward according to the example given above if no role playing adventure is determined and acted out. At least one of the removable pegs 15 lands in at least one of the plurality of holes 20 in accordance with the predetermined number associated with the color of the side of the gaming member 18 facing in an upward direction. A kissing shield game winning player is determined based upon the first person playing the kissing shield game to advance his/her removable peg 15 in a forward direction along the plurality of holes 20 in the predetermined pattern on the playing surface 16 until the plurality of holes 20 in the predetermined pattern ends.

In view of the above, it will be seen that the several objects of the invention are achieved and that other advantageous results are attained. As various changes could be made in the above product and method without departing from the scope of the invention, it is intended that all matter contained in the above description or shown in the accompanying drawings shall be interpreted as illustrative and not in a limiting sense.

It should be noted that the foregoing drawings and accompanying descriptions are intended to be exemplary of several preferred embodiments of the invention and are not exhaustive of the possibilities of the types of sheilds within the intended scope of the invention. It should also be understood that modifications will readily occur to those skilled in the art within the spirit of the invention. Such modifications could include using different color materials on each side of the membrane 10 or sheet to ensure that the user consistently originates his kisses from the same side of the membrane 10. The membrane 10 could also be made of a plastic material which would be unaffected by products, such as foundation, lipstick, petroleum jelly, and other cosmetics routinely used by most women.

What is claimed is:

1. A finger kissing shield for preventing an exchange of microorganisms between a user and an object during an act of kissing, the finger kissing shield comprising:
a thin, flexible membrane substantially impervious to microorganisms, the thin flexible membrane having a first cheek portion for substantially covering a first cheek area of the user, a second cheek portion for substantially covering a second cheek area of the user, a clearance between the first cheek portion and the second cheek portion for accommodating the nose of the user, a chin portion for covering the chin of the user, a central portion for covering the lips and mouth of the user, wherein the plurality of thin, flexible membranes have a pocket sized and configured so as to be positionable over at least a portion of a user's first finger and a user's second finger of the user's hand so as to create a space between the fingers and extend the first cheek portion and the second cheek portion in opposite lateral directions for disposing the thin flexible membrane between the user and the object being kissed such that the thin flexible member thereby prevents the exchange of microorganisms between the user and the object during the act of kissing.

2. The shield according to claim 1, wherein the first finger and the second finger is selected from the group consisting of an index finger, a middle finger, a ring finger, a pinky finger, and a thumb.

3. The shield according to claim 1, wherein the plurality of thin, flexible membranes has at least two separate pockets whereby one pocket accepts the first finger and the other pocket accepts the second finger and a single pane of membrane extends between the at least two pockets.

4. The shield according to claim 1, wherein the central portion of the thin flexible member includes a protruding portion, the protruding portion configured to substantially accommodate the tongue of the user such that the the user's tongue is positionable in the protruding portion.

5. The shield according to claim 1, wherein the thin flexible membrane further includes a first side and a message or a design printed on the first side of the thin, flexible membrane.

6. The shield according to claim 1, wherein said thin, flexible membrane is constructed of a synthetic material.

7. The shield according to claim 1, wherein said thin, flexible membrane is constructed of latex.

8. The shield according to claim 1, wherein said thin, flexible membrane is constructed of a material selected from the class of materials consisting of polyethylene, vinyl or polypropylene.

9. The shield according to claim 1, wherein a first side of the thin, flexible membrane is a first color and the second side of the thin flexible membrane is a second color.

10. The method of preventing transmission of germs and the like during kissing consisting of imposing a thin, flexible membrane substantially impervious to microorganisms in front of the lips of a user, the thin, flexible membrane having a first cheek portion for substantially covering a first cheek area of the user, a second cheek portion for substantially covering a second cheek area of the user, a clearance between the first cheek portion and the second cheek portion for accommodating the nose of the user, a chin portion for covering the chin of the user, a central portion for covering the lips of the user, wherein the plurality of thin, flexible membranes have a pocket sized and configured so as to be positionable over at least a portion of a user's first finger and a user's second finger of the user's hand so as to create a space between the fingers and extend the first cheek portion and the second cheek portion in opposite lateral directions for disposing the thin flexible membrane between the user and the object being kissed such that the thin flexible member thereby prevents the exchange of microorganisms between the user and the object during the act of kissing.

11. A kissing shield game, comprising:
a plurality of removable pegs;
a playing surface having a plurality of holes, wherein each of the plurality of holes is capable of receiving at least one of the plurality of removable pegs, the plurality of holes further being arranged in a predetermined pattern;
a gaming member having at least two sides, wherein each of the sides of the gaming member has a different color associated therewith as well as a predetermined set of alphanumeric symbols associated therewith;
a plurality of thin, flexible membranes substantially impervious to microorganisms, each of the plurality of thin, flexible membranes having a first cheek portion for substantially covering a first cheek area of a user, a second cheek portion for substantially covering a second cheek area of the user, a clearance between the first cheek portion and the second cheek portion for accommodating the nose of the user, a chin portion for covering the chin of the user, a central portion for covering the lips of the user, wherein the plurality of thin, flexible membranes have a pocket sized and configured so as to be positionable over at least a portion of a user's first finger and a user's second finger of the user's hand so as to create a space between the fingers and extend the first cheek portion and the second cheek portion in opposite lateral directions for disposing the thin flexible membrane between the user and the object being kissed such that the thin flexible member thereby prevents the exchange of microorganisms between the user and the object during the act of kissing; and
a resettable timer.

12. The kissing shield game according to claim 11, wherein the first finger and the second finger is selected from the group consisting of an index finger, a middle finger, a ring finger, a pinky finger, and a thumb.

13. The kissing shield game according to claim 11, wherein the plurality of thin, flexible membranes has at least two separate pockets whereby one pocket accepts the first finger and the other pocket accepts the second finger and a single pane of membrane extends between the at least two pockets.

14. The kissing shield game according to claim 11, wherein each color associated with one of the sides of the gaming member is assigned a predetermined numerical value.

15. The kissing shield game according to claim 11, further comprising a set of rules which govern the function of the kissing shield game and the order and actions of a kissing shield game participant.

16. The kissing shield game according to claim 11, wherein a plurality of persons may be divided into a plurality of teams for playing the kissing shield game.

17. The kissing shield game according to claim 11, wherein each of the plurality of thin, flexible membranes is colored and at least one of the removable pegs is colored to correspond to the color of at least one of the plurality of thin, flexible membranes.

18. A method for teaching safe affection to at least one person playing a kissing shield game, the method comprising the steps of:
providing a kissing shield game, comprising;
a plurality of removable pegs;
a playing surface having a plurality of holes, wherein each of the plurality of holes is capable of receiving at least one of the plurality of removable pegs, the plurality of holes further being arranged in a predetermined pattern;
a gaming member having at least two sides, wherein each of the sides of the gaming member has a different color associated therewith as well as a predetermined set of alphanumeric symbols associated therewith;
a plurality of thin, flexible membranes substantially impervious to microorganisms, each of the plurality of thin, flexible membranes having a first cheek portion for substantially covering a first cheek area of a user, a second cheek portion for substantially covering a second cheek area of the user, a clearance between the first cheek portion and the second cheek portion for accommodating the nose of the user, a chin portion for covering the chin of the user, a central portion for covering the lips of the user, wherein the plurality of thin, flexible membranes have a pocket sized and configured so as to be positionable over at least a portion of a user's first finger and a user's second finger of the user's hand so as to create a space between the fingers and extend the first cheek portion and the second cheek portion in opposite lateral directions for disposing the thin flexible membrane between the user and the object being kissed such that the thin flexible member thereby prevents the exchange of microorganisms between the user and the object during the act of kissing and the thin, flexible member constructed of a material so as to be impervious to microorganisms and preventing exchange of microorganisms between the person and an object while engaged in the act of kissing; and
a resettable timer;
placing at least one of the thin, flexible members over the hand of the user so as to form a finger kissing shield;
throwing the gaming member along the playing surface so that one of the sides lands facing in an upward direction;
determining a role playing adventure within a predetermined time so as to teach safe affection utilizing the finger kissing shield as a means to block contact between the person and an object in accordance with the alphanumeric symbol associated with the side of the gaming member facing in an upward direction, the end of the predetermined time being signaled by the resettable timer;
determining a number for sequential movement of at least one of the plurality of removable pegs in accordance with a predetermined number associated with the color of the side of the gaming member facing in the upward direction; and
landing in at least one of the plurality of holes in accordance with the predetermined number associated with the color of the side of the gaming member facing in an upward direction.

19. The method of claim 16, further including the step of kissing a selected person or object determined by the player with the finger kissing shield so that at least one person playing the kissing shield game learns safe affection and the proper use of the finger kissing shield through repetition.

20. The method of claim 16, further including the step of moving at least one of the removable pegs in a forward direction along the plurality of holes arranged in the predetermined pattern in accordance with the predetermined number associated with the color of the side of the gaming member facing in an upward direction.

21. The method of claim 16, further including the step of moving at least one of the removable pegs in a backward direction along the plurality of holes arranged in the predetermined pattern in accordance with the predetermined number associated with the color of the side of the gaming member facing in an upward direction upon the person playing the kissing shield game failing to determine the role playing adventure within a specified time.

22. The method of claim 16, further including the step of determining a kissing shield game winning player based upon the first person playing the kissing shield game to advance in a forward direction along the plurality of holes in the predetermined pattern on the playing surface until the plurality of holes in the predetermined pattern ends.
